(19) United States
(12) Patent Application Publication
(10) Pub. No.: US 2008/0299105 A1
(43) Pub. Date: Dec. 4, 2008

(54) Inhibition of Feline Calicivirus

(76) Inventor: Runtao He, Manitoba (CA)

Correspondence Address: ADE & COMPANY INC., 2157 Henderson Highway, WINNIPEG, MB, R2G1P9, CA

(21) Appl. No.: 11/572,282

(22) PCT Filed: Sep. 13, 2005
(86) PCT No.: PCT/CA2005/001384

§ 371 Date: Apr. 9, 2007

Related U.S. Application Data
(60) Provisional application No. 60/608,863, filed on Sep. 13, 2004.

(51) Int. Cl. A61K 38/50; A61P 31/12

(52) U.S. Cl. 424/94.6

(57) ABSTRACT

We conducted feline calicivirus (FCV) inhibition experiment with two anti-viral aminohydrolases, asparaginase and glutaminase. We found that in the presence of 8 units and 4 units/ml of asparaginase, about 90% of FCV replication was inhibited. In contrast, glutaminase showed no significant inhibition effect on the virus replication. We have also shown that asparaginase did not inhibit the replication of adenovirus suggesting that the inhibition was specific. Our results implicated that asparaginase could be used as a candidate for anti-FCV drug development.

### BACKGROUND OF THE INVENTION

The feline calicivirus (FCV) occurs globally and infects all breeds of cats, characterized by upper respiratory symptoms, pneumonia, oral ulceration and arthritis [1]. Even though vaccination has reduced the incidence of clinical disease, the prevalence of the virus has not decreased significantly. Recent reports indicated that new strains of FCVs are increasingly causing a highly contagious febrile hemorrhagic syndrome. The strain causing this syndrome is different from the vaccine strain, thus new vaccines or viral replication inhibitors of FCV may be required to prevent the spread of the virus [2].

FCV is a non-enveloped, single-stranded, positive-sense RNA virus that is classified in the genus Vesivirus of Caliciviridae. The genome of the virus is about 7.5 kb, while a subgenomic RNA about 2.2-2.4 kb in size is also packed in virions [3,4]. The major FCV structural proteins are VP1, which is cleaved from a precursor protein, the 14 kD leader of the capsid (LC), and VP2. There are also several non-structural proteins ranging from 13 kD to 96 kD [3,5,6].

Up until now, no known inhibitors has been characterized that block the replication of the virus [7,8]. Amidohydrolases have been used for antiviral candidates for many viruses [9, 10], for example HIV [11]. In the current analysis we tried two amidohydrolases, asparageinase and glutaminase, to characterize their potential as anti-FCV agents.

### SUMMARY OF THE INVENTION

According to a first aspect of the invention, there is provided a method of treating or preventing a calicivirus infection, comprising administering to an animal in need of such treatment an effective amount of asparaginase.

### BRIEF DESCRIPTION OF THE DRAWINGS

FIG. 1. Inhibition of FCV by asparaginase. CFRK cells were infected with and without FCV and treated with 8 units/ml of asparaginase and glutaminase. After 24 h, cells were photographed.

FIG. 2. Dose dependent assay on asparaginase inhibition of FCV replication. CFRK cells were infected with FCV and treated with serial dilutions of aspraginase. 24 h. post infection, cells were stained with 0.1% crystal violet and 5% formaldehyde.

FIG. 3. Adenovirus inhibition assay with asparaginase HEK293 cells were transfected with andenovirus X construct expressing EGFP and treated with 8 units/ml of aspraginase. After 24 h., cells were visualized under a fluorescent microscope.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein are feline calicivirus (FCV) inhibition experiment conducted with two anti-viral aminohydrolases, asparaginase and glutaminase. We found that in the presence of 8 units and 4 units/ml of asparaginase, about 90% of FCV replication was inhibited. In contrast, glutaminase showed no significant inhibition effect on the virus replication. We have also shown that asparaginase did not inhibit the replication of adenovirus, suggesting that the inhibition was specific. The results indicate that asparaginase could be used as a treatment for animals infected with FCV as well as a candidate for anti-FCV drug development. For example, structural equivalents to regions of asparaginase as well as chemically or otherwise modified forms of asparaginase could be screened for effectiveness as a calicivirus inhibitor.

As will be appreciated by one of skill in the art, infections caused other members of the calicivirus family, for example, but by no means limited to human calicivirus or rabbit calicivirus, may be treated or prevented with an effective amount of asparaginase, as discussed below.

As used herein, “effective amount” in regards asparginase refers to an amount that is sufficient to reduce FCV replication, for example, by reducing the rate of viral replication, by reducing viral load or viral burst size or by reducing the rate of replication initiation, compared to an untreated control. As will be appreciated by one of skill in the art, this amount may vary according to the age, weight and condition of the patient and the exact amount can be determined by one of skill in the art through routine experimentation. In some embodiments, an effective amount may be 0.1 units/ml to 100 units/ml, or 1 unit/ml to 50 units/ml or 1 unit/ml to 10 units/ml.

In some embodiments, asparaginase at concentrations or dosages discussed above may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

In some embodiments, an effective amount of asparaginase is administered to an animal, for example, a feline, for example, a cat, that is infected with, suspected of being infected with or at risk of developing an infection from feline calicivirus. Because asparaginase inhibits replication of feline calicivirus, it will accomplish at least one or more of the following: reduce viral load, prevent infection or reinfection, reduce spread of virus, or ameliorate one or more symptoms associated with a feline calicivirus.

In some embodiments, an effective amount of asparaginase is administered to an animal, for example, a human, for example, that is infected with, suspected of being infected with or at risk of developing an infection from human calicivirus. Because asparaginase inhibits replication of feline calicivirus, it will accomplish at least one or more of the following: reduce viral load, prevent infection or reinfection, reduce spread of virus, or ameliorate one or more symptoms associated with a feline calicivirus.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

The invention will now be described by examples; however, the invention is not limited to the examples.

Cell Culture and cell staining—FCV was from Dr. Michael Carpenter, the virus showed 97% identity to the reported strain with accession number NC_{—}001481 (Carpenter et al, unpublished data). The Feline kidney cell line CFRK cells were cultured in Minimum Essential Medium, supplemented with 10% heat-inactivated fetal bovine serum (Inivitrogen, Carlsbad Calif.), and 1% penicillin/streptomycin. CFRK cells have been shown to be susceptible to FCV infection (4). All cell cultures were maintained in a humidified 5% CO₂ incubator at 37° C. The asparaginase (Sigma, St. Louis, Mo.) was added to the medium samples in serial dilutions comprised of 8 units/ml, 4 units/ml, 2 units mg/ml and 1 units/ml. The same dilutions of glutaminase were also used in the above assay.

Cell staining—the cell staining was performed using were performed 20 h. post the infection using procedures as described [12.], briefly, 24 h post infection, cells were fixed with 5% formaldehyde and stained with 0.1% crystal violet.

Inhibition of Adenovirus expressing EGFP—Asparaginase was used in a comparative study for inhibition of the adenovirus Adeno X, which carries a replication reporter gene expressing EGFP (Clontech, Palo Alto, Calif.). The HEK293 cells were infected with Adv-EGFP at a moi of 1. Virus replication was visualized by the presence of EGFP fluorescence using Zeiss M200 Microscope.

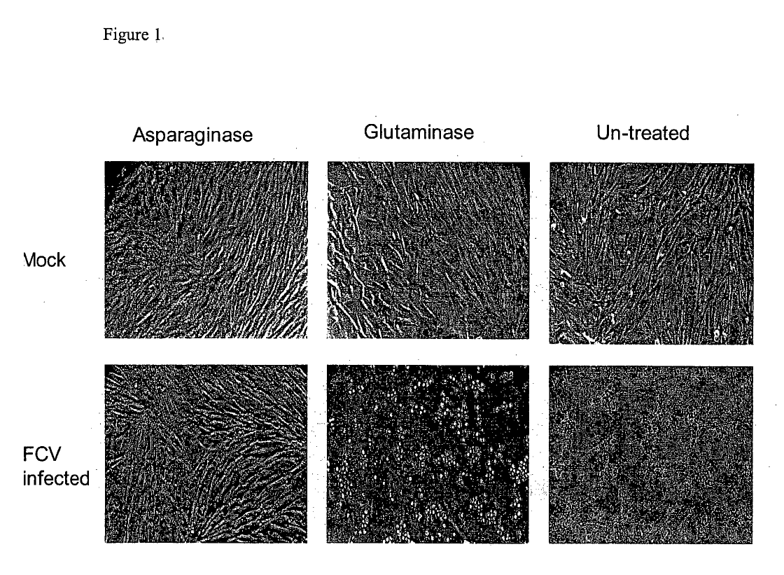

Asparaginase inhibits FCV replication in CFRK cells. We have tested the anti-FCV effect of asparagninase over a range of concentrations, from 8 units/ml prepared in Minimum Essential Medium with 10% of fetal calf serum to 1 unit/ml. Twenty hours post infection; cell morphologies in all groups were photographed (FIG. 1a). As shown in FIG. 1, there were significant cytopathic effects (CPE) in untreated and glutaminase-treated cells infected with FCV, the asparagines-treated cells only showed minor CPE, over 90 percent of the cells were protected by the treatment of asparaginase. We also added glutaminase and asparaginase to the mock-infected cells to determine the cytotoxic effects of the aminohydrolases. As shown in FIG. 1, there was no significant cytoxicity from the two aminohydrolases, confirming that the CPE was due to FCV replication.

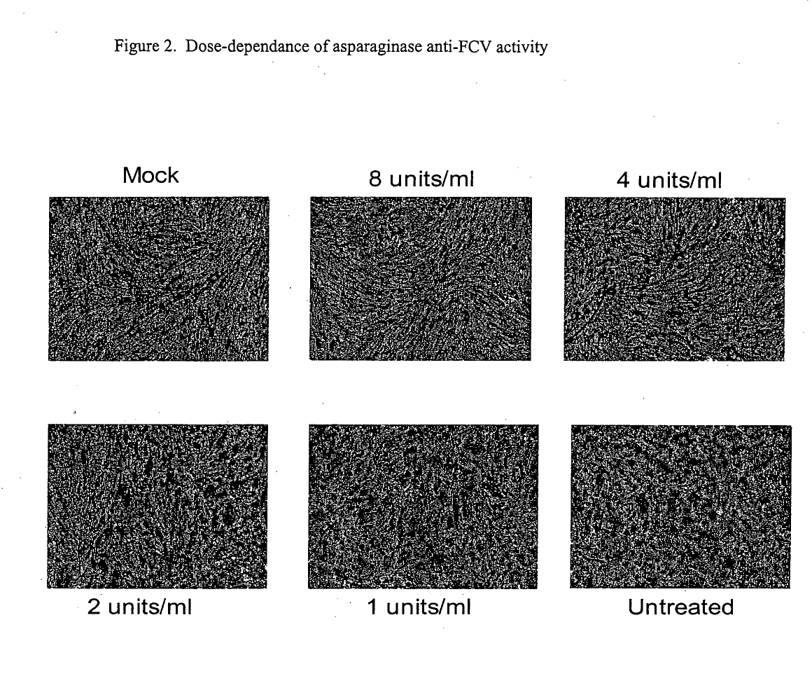

Dose dependent analysis of asparaginase against FCV—we performed the above inhibition analysis with serial dilutions of aspraginase. As shown in FIG. 2, compared with the untreated sample, cells treated with 8 units/ml of aspraginase had the least amount of CPE, the inhibition effect decreases following the decrease of asparaginase concentration; at 1 units/ml, asparaginase still showed mild level of inhibition on CPE. This result implicated that the antiviral effect of asparagines was dose-dependent.

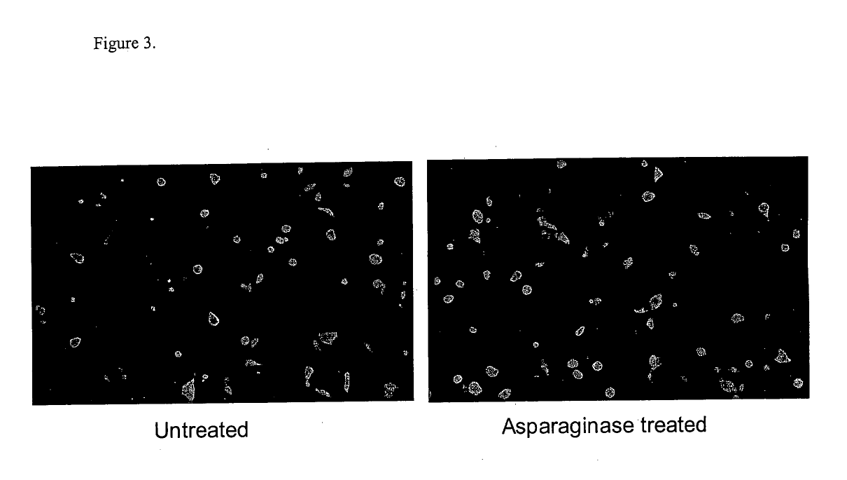

Inhibition of asparaginase to adenovirus—To characterize the specificity of anti-viral activity of asparaginase, we also tested the compound for its ability to block the infection of adenovirus replication. The replication deficient adenovirus type 5, missing E1 and E3 genes were used to infect HEK-293 cells. The same concentration of asparaginase used in the abovementioned FCV inhibition experiment was added to the adenovirus-infected cells. As shown in FIG. 3, compared with untreated cells, adenovirus replication was virtually not affected by the treatment of asparaginase.

We have shown for the first time that asparaginase could inhibit the replication of feline calicivirus. Although FCV generally causes mild disease, virus is highly infectious and is a major cause of morbidity and even mortality among cat breeds [13].

FCV is a member of small round structured viruses (SRSVs), a group of Caliciviruses that cause gastroenteritis. In recent years SRSVs have emerged to be one of the major public concerned pathogen group [14,15]. The transmissions of SRSVs are through close contact, contaminated food and aerosols; moreover, SRSVs have been shown to be heat and ether resistant and acid stable [16,17]; these factors make it difficult to control the transmission of the virus particles. For the above reason, searching for anti-viral candidates that inhibit the virus replication can contribute the reduction of prevalence and the treatment of diseases.

The antiviral activity of asparagines has been reported previously, for example, it was shown that asparaginase could inhibit the replication of HIV[11]. The exact mechanism of the inhibition of FCV replication by asparaginase is yet to be characterized. Asparaginase catalyses the hydrolysis of asparagines to aspartic acid and ammonia [18], it has been widely used for anti-cancer therapies, especially anti-lacute lymphatic eukemia therapies [19,20]. The discrepancy between the potency of glutaminase and asparaginase is not yet understood, it may be possible that asparagines is crucial in forming conformational function epitops for FCV replication.

In conclusion, for the first time we found that asparaginase has potent anti-viral effect against FCV infection, suggesting that it could posses therapeutic value for the treatment of FCV infection.

### REFERENCES

1. Hurley K F, Sykes J E. Update on feline calicivirus: new trends. Vet Clin North Am Small Anim Pract. 2004. 33(4):759-72
2. Baulch-Brown C, Love D N, Meanger J. Feline calicivirus: a need for vaccine modification? Aust Vet J. 1997. 75(3):209-13.
3. Thiel H J and Konig M. Caliciviruses: an overview. Vet Microbiol. 1999. 69(1-2):55-62.
4. Clarke I N and Lambden P R. Organization and expression of calicivirus genes. J Infect Dis. 2000. 181 S309-16
5. Ohlinger V F, Haas B, Thiel H J. Rabbit hemorrhagic disease (RHD): characterization of the causative calicivirus. Vet Res. 1993;24(2):103-16
6. Cubitt W D. Human, small round structured viruses, caliciviruses and astroviruses. Baillieres Clin Gastroenterol. 1990 September;4(3):643-56.
7. Pesavento P A, MacLachlan N J, Dillard-Telm L, Grant C K, Hurley K F. Pathologic, immunohistochemical, and electron microscopic findings in naturally occurring virulent systemic feline calicivirus infection in cats. Vet Pathol. 2004. 41(3):257-63.
8. Umehashi M, Imamura T, Akiyama S, Matsuda J, Tokiyoshi S, Tohya Y et al. Pre-exposure treatment of cats with anti-FHV-1 and anti-FCV mouse-cat chimeric antibodies. J Vet Med Sci. 2003. 65(5):563-6.
9. Thiry L and Cappel R. Antiviral effect of asparaginase and of Burkitt cell extracts in combination with other drugs. Biomedicine. 1974. 20(3):198-204.
10. Maral R and Werner G H. Antiviral activity of L-asparaginase. Nat New Biol. 1971 232(2):187-8.
11. Avramis V I, Kwock R, Avramis I A, Cohen L J, Inderlied C. Synergistic antiviral effect of PEG-asparaginase (ONCASPAR), with protease inhibitor alone and in combination with RT inhibitors against HIV-1 infected T-cells: a model of HIV-1-induced T-cell lymphoma. In Vivo. 15(1):1-9.
12. Gomwalk N E. A direct plaque assay for Mount Elgon bat virus. J Virol Methods. 1980; 1(4):201-8
13. Radford A D, Dawson S, Gaskell R M, Foley J, Hurley K, Pedersen N C. Haemorrhagic fever, oedema and high mortality associated with FCV infection. Vet Rec. 2002;151(5):155
14. Chadwick P R, Beards G, Brown D, Caul E O, Cheesbrough J, Clarke I, et al. Management of hospital outbreaks of gastro-enteritis due to small roundstructured viruses. J Hosp Infect. 2000. 45(1):1-10.
15. Schreier E, Doring F, Kunkel U. Molecular epidemiology of outbreaks of gastroenteritis associated with small round structured viruses in Germany in 1997/98. Arch Virol. 2000;145(3):443-53.
16. Clarke I N, Lambden P R. iral zoonoses and food of animal origin: caliciviruses and human disease. Arch Virol Suppl. 1997;13:141-52
17. Caceres V M, Kim D K, Bresee J S, Horan J, Noel J S, Ando T et al. A viral gastroenteritis outbreak associated with person-to-person spread among hospital staff. Infect Control Hosp Epidemiol. 1998; 19(3):162-7.
18. Balis M E. Determination of glutamic and aspartic acids and their amides. Methods Biochem Anal. 1971;20:103-33.
19. Pinheiro J P and Boos J. The best way to use asparaginase in childhood acute lymphatic leukaemia—still to be defined? Br J Haematol. 2004. 125(2):117-27
20. Muller H J and Boos J. Use of L-asparaginase in childhood ALL. Crit Rev Oncol Hematol. 1998; 28(2):97-113

While the preferred embodiments of the invention have been described above, it will be recognized and understood that various modifications may be made therein, and the appended claims are intended to cover all such modifications which may fall within the spirit and scope of the invention.

1. A method of treating or preventing a calicivirus infection, comprising administering to an animal in need of such treatment an effective amount of asparaginase.

2. The method according to claim 1 wherein the calicivirus is human calicivirus and the animal is a human.

3. The method according to claim 1 wherein the calicivirus is feline calicivirus and the animal is a feline.

4. The method according to claim 1 wherein the calicivirus is rabbit calicivirus and the animal is a rabbit.
